(19) United States
(12) Patent Application Publication
(10) Pub. No.: US 2006/0185196 A1
(43) Pub. Date: Aug. 24, 2006

(54) Massage shoes

(76) Inventor: Dong-lei Wang, Zuhai City (CN)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(21) Appl. No.: 11/354,686

(22) Filed: Feb. 15, 2006

(30) Foreign Application Priority Data
Feb. 19, 2005 (CN) 200520054968.9

(51) Int. Cl. A61F 5/14

(52) U.S. Cl. 36/141

(57) ABSTRACT

The present invention provides a massage shoe, comprising a sole, a shoe body fixed on said sole, and a massage assembly; wherein a concave is formed on said sole for housing said massage assembly, said massage assembly having at least two massage bodies made from deformable materials, and wherein at least one massage protrusion is disposed on the top of each of said massage bodies, and a cavity is formed inside each of said massage bodies; and wherein said cavities of said massage bodies communicate with each other via pipelines, and said cavities and pipelines form a connected and sealed chamber filled with a fluid; wherein said chamber is partially filled, so that said fluid may flow within cavities of different massage bodies.

CROSS-REFERENCE TO RELATED APPLICATION

This application claims priority of Chinese Patent Application No. 200520054968.9 filed on Feb. 19, 2005, entitled “Massage Shoes”, which is incorporated herein by reference for all purposes.

1. Technical Field

The present invention relates to massage shoes, more particularly, to shoes having protrusions for massage.

### BACKGROUND OF THE INVENTION

At present, with the improvement of people's consciousness on health care, traditional shoes can no longer satisfy the higher demands of people, and shoes with a health care effect are popular for users. In the prior art, one kind of shoes with a health care effect are made by adding a medicinal pad to a traditional shoe, which have an assistant efficacy for treatment; however, this kind of shoes work only as a temporary solution. Another kind of shoes with a health care effect are massage shoes. Typically, massage shoes in the market have protrusions fixed on the soles of the shoes, wherein the protrusions produce a massage effect for a foot bottom while a person walks. However, since the locations of the protrusions are fixed, the pressure distribution is not uniform and the massage effect is not satisfactory. Moreover, the locations of the protrusions are required to be accurate, otherwise, these protrusions cannot release fatigue from a user, let alone produce a treatment effect. Furthermore, the shoes may easily get locally distorted after long time use, which affects the effects of treatment and health care.

### SUMMARY OF THE INVENTION

The object of the present invention is to provide massage shoes, which provide a more evenly distributed pressure, have a better massage effect, and make users feel more comfortable.

A massage shoe according to the present invention, comprising a sole, a shoe body fixed on said sole, and a massage assembly; wherein a concave is formed on said sole for housing said massage assembly, said massage assembly having at least two massage bodies made from deformable materials, and wherein at least one massage protrusion is disposed on the top of each of said massage bodies, and a cavity is formed inside each of said massage bodies; and wherein said cavities of said massage bodies communicate with each other via pipelines, and said cavities and pipelines form a connected and sealed chamber filled with a fluid. Said chamber is partially filled, so that said fluid may flow within cavities of different massage bodies.

Said massage protrusions are disposed corresponding to acupuncture points in the foot bottom.

A gas or a liquid or a flexible and deformable solid can serve as said fluid.

When not pressed, the fluid remains in the cavities of the massage bodies. When one of said massage bodies is pressed by a foot bottom, the fluid in the cavity of the pressed massage body flows to the cavity of other massage body via pipelines under the stress from a foot bottom. Accordingly, the massage bodies are deformed and massage the corresponding positions in the foot bottom by said at least one protrusion disposed on the top of the massage bodies

With the massage shoes according to the present invention, the pressure can be relatively uniformly distributed onto a plurality of massage bodies, so that the massage effect can be improved, and a local distortion of the massage shoes is not easy to happen.

### BRIEF DESCRIPTION OF THE DRAWINGS

FIG. 1 is a schematic view showing the structure of a sole of one embodiment of the massage shoes according to the present invention;

FIG. 2 is a sectional view of the sole as shown in FIG. 1.

### DETAILED DESCRIPTION OF THE PREFERRED EMBODIMENTS

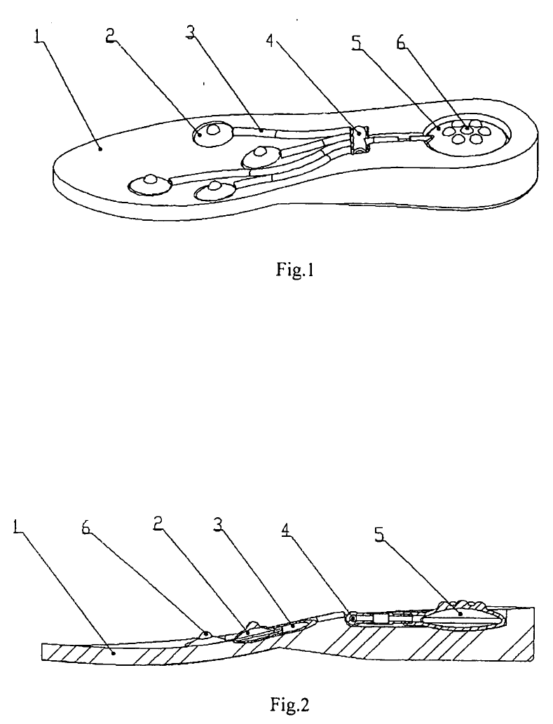

As shown in FIG. 1 and FIG. 2, a massage shoe of the present invention comprises a sole 1, a shoe body (not shown) fixed on the sole 1, and a massage assembly. A concave is formed on the sole 1 for housing the massage assembly.

The massage assembly includes a big massage body 5 located in the middle of the heel of the sole, and four small massage bodies 2 located in the front of the sole 1. The big massage body or the small massage bodies is in the shape of a hollow discus, and is flexible and deformable, which can be made of rubber, plastic, or complex leather and the like. Each of the small massage bodies has one massage protrusion on its top positioned corresponding to the acupuncture point in the foot bottom. The big massage body 5 has a plurality of massage protrusions 6 on its top positioned corresponding to the acupuncture point in the foot bottom. A distributor 4 is provided between the big massage body 5 and the small massage bodies 2, and a pipeline 3 is connected between each of the cavities and the distributor 4, so as to form a connected and sealed chamber. The pipelines 3 are made of elastic and contractive materials, such as rubber, plastic, or complex leather and the like. Said chamber is partially filled, so that said fluid may flow within cavities of different massage bodies.

A gas or a liquid or a flexible and deformable solid can serve as said fluid.

According to the known traditional Chinese medicine, the internal organs of men have corresponding acupuncture points, namely reflex areas, in the foot bottom. Through massage, the corresponding reflex areas will be stimulated. In this way, the blood circulation of the corresponding organ will be better. Therefore, it will improve the endocrine system, immunity of one's body and recover it's function. The quantity and position of the massage body and the protrusion thereon may be arranged according to desire of treatment and health care.

For example, the massage assembly may include a big massage body 5 located in the middle of the heel of the sole, and two small massage bodies 2 located in the arch and the middle part of the front sole of the foot, corresponding to the reflex areas of the kidney and the lung of a human's body, respectively. Each of the small massage bodies 2 has one massage protrusion on its top positioned corresponding to an acupuncture point. The big massage body 5 has a plurality of massage protrusions 6 on its top positioned corresponding to the acupuncture points. Thus it will help treatment or health care for kidney and lung.

Alternatively, the massage assembly may include a big massage body 5 located in the middle of the heel of the sole, and three small massage bodies 2 located in the sole under the roots of the big toe, the middle toe and the small toe corresponding to the reflex areas of the brain, the eye and the ear of a human's body, respectively. Each of the small massage bodies 2 has one massage protrusion on its top positioned corresponding to an acupuncture point. The big massage body 5 has a plurality of massage protrusions 6 on its top positioned corresponding to the acupuncture points. Thus it will help treatment or health care for brain, eye and ear.

When not pressed, the fluid remains in the cavities of the massage bodies. In use, a pressure will be produced to some position of the sole 1 as long as there is movement on the foot of the user wearing the massage shoes. When one of the massage bodies is pressed by a foot bottom, the fluid in the cavity of the pressed massage body flows to the cavity of other massage body via pipelines under the stress from a foot bottom. Accordingly, other massage bodies expand and massage the corresponding positions in the foot bottom by the protrusions disposed on the top of the massage bodies. Along with the movement of the foot, these expanded massage bodies are compressed and pass a pressure to other massage bodies. The formerly compressed massage bodies are made to expand and massage the corresponding positions in the foot bottom by the protrusions disposed on the top of the massage bodies. So on, the plurality of acupuncture points in the foot bottom can be massaged.

What is claimed is:

1. A massage shoe according to the present invention, comprising a sole, a shoe body fixed on said sole, and a massage assembly; wherein a concave is formed on said sole for housing said massage assembly, said massage assembly having at least two massage bodies made from deformable materials, and wherein at least one massage protrusion is disposed on the top of each of said massage bodies, and a cavity is formed inside each of said massage bodies; and wherein said cavities of said massage bodies communicate with each other via pipelines, and said cavities and pipelines form a connected and sealed chamber filled with a fluid; wherein said chamber is partially filled, so that said fluid may flow within cavities of different massage bodies.

2. The massage shoe according to claim 1, wherein said massage protrusions are located in correspondence with the acupuncture points in the foot bottom.

3. The massage shoe according to claim 1, wherein a gas, a liquid or a flexible and deformable solid may serve as said fluid.

4. The massage shoe according to claim 1, wherein said massage body is in the shape of a hollow discus.

5. The massage shoe according to claim 1, wherein said massage body is made of rubber, plastic, or complex leather.

6. The massage shoe according to claim 1, wherein said pipelines are made of rubber, plastic, or complex leather.

7. The massage shoe according to claim 1, wherein said massage assembly includes a big massage body located in the middle of the heel of the sole, and at least two small massage bodies located in the front of the sole; and wherein a distributor is provided between the big massage body and the small massage bodies, and a pipeline is connected between each of the cavities and the distributor, so as to form a connected and sealed chamber.

8. The massage shoe according to claim 7, wherein said massage assembly includes a big massage body located in the middle of the heel of the sole, and two small massage bodies located in the arch and the middle part of the front sole of the foot, corresponding to the reflex areas of the kidney and the lung of a human's body, respectively.

9. The massage shoe according to claim 7, wherein said massage assembly includes a big massage body located in the middle of the heel of the sole, and three small massage bodies located in the sole under the roots of the big toe, the middle toe and the small toe corresponding to the reflex areas of the brain, the eye and the ear of a human's body, respectively.
